(19) United States
(12) Patent Application Publication
(10) Pub. No.: US 2014/0128802 A1
(43) Pub. Date: May 8, 2014

(54) Chemical Applicator

(71) Applicant: Rad Omid, St Georges (AU)

(72) Inventor: Rad Omid, St Georges (AU)

(73) Assignee: ARIANA HOLDINGS PTY LTD, ADELAIDE (AU)

(21) Appl. No.: 14/153,270

(22) Filed: Jan. 13, 2014

Related U.S. Application Data
(63) Continuation-in-part of application No. 12/097,325, filed on Jun. 20, 2008, now Pat. No. 8,663,176.

(30) Foreign Application Priority Data
Dec. 14, 2005 (AU) 2005907011

(51) Int. Cl. A01G 7/00; A01G 1/00; A61M 37/00

(52) U.S. Cl. CPC A01G 7/00; A61M 37/00; A01G 1/001
USPC 604/47; 47/1.5; 47/58.1 R

(57) ABSTRACT

The present invention relates to a device and method for a chemical applicator used applying chemicals to living organism's internal tissue, including the internal tissue of plants and animals, for organisms which reside on or in mediums such as land, air or water. The device of the present invention is predicated, in part, on the piercing elements being engageable with the chemical reservoir in order to effect the coating of the piercing elements with the chemical. Once coated, these piercing elements are then used to pierce the living organism's tissue such as plant tissue to deliver the chemical.

### FIELD OF THE INVENTION

The present invention relates to the field of chemical applicators and, specifically to the subfield of chemical applicators which incorporate chemical application devices and methods for applying chemicals to the internal tissue of living organisms, including the internal tissue of plants and animals which may reside in media including air, land and/or water. The chemical applicator device and method is designed to increase the effectiveness of chemicals applied to the body tissues of target organisms, improving the effectiveness of the chemicals at lower doses than achieved with conventional devices and methods of application, whilst minimising exposure of non-target animals or plants to the chemical; however, the invention is not restricted to this particular field of use.

### BACKGROUND OF THE INVENTION

Methods and devices for applying chemicals, such as herbicides, pesticides, fungicides and fertilisers, to plants are known. Such methods and devices include spraying and direct application methods and devices.

It is known, for example, that spraying-based methods may be suitable when large areas of target plants are involved. However, spraying methods may be unsuitable where target plants are interspersed with non-target plants. In this case, spraying-based methods of chemical application may not be sufficiently accurate to apply a chemical only to the target plants. This may result in damage of nearby non-target plants and contamination of the target plant environs.

Furthermore, spraying methods may also not be suitable under particular environmental conditions such as high wind, rain, air/soil temperatures, humidity extremes and/or in aquatic environments. Under these conditions, the chemical may be either dispersed or diluted such that the concentration on the target plants and/or the chemical may wash off or drift onto non-target plants and cause damage by reducing the efficacy of the chemical or rendering it ineffective altogether.

In light of the problems associated with spraying-based methods, a number of direct-application or contact type chemical applicator technologies have been developed in various attempts to address these problems.

For example, U.S. Pat. No. 4,357,779, to M. E. Maddock, describes a chemical applicator comprising a sponge applicator connected to a chemical reservoir wherein flow of chemical from the reservoir to the sponge applicator is controlled by a flow control valve.

U.S. Pat. No. 4,403,881, to J. H. Keeton, describes a hollow handled chemical applicator which comprises a chemical reservoir within the upper part of the handle. Chemical from the reservoir is supplied in discrete doses to a sponge applicator at the lower end of the device through a slit valve, which is openable in response to the operation of a button by a user of the device.

U.S. Pat. No. 4,716,677, to J. E Moore, describes a hand-held chemical applicator comprising a chemical applicator pad mounted between pincers or tweezers. The pincers or tweezers may then be used to grip a plant and thereby apply the herbicide to the surface of the plant gripped by the pincers.

Each of the applicators described above uses a sponge or absorbent pad to apply chemical to the surface of a plant. As such, the chemical is subject to wash-off during rainfall. This wash-off potentially leads to exposure of non-target plants or animals to the chemical and/or unnecessary contamination of the environment with the chemical. In areas with relatively high or low air and/or soil temperatures as well as high humidity tropical and sub-tropical areas, the chemicals are not as effective, so the external application of such chemicals is fraught with problems in these conditions.

Furthermore, wash-off potentially:
1. exposes non-target plants and animals to the chemical;
2. reduces the concentration of the chemical on the target surface; and/or
3. damages soil thus potentially causing harm to living organisms within the soil.

This potentially reduces the effective dose of the chemical delivered to the target plant. The reduced effective dose of the chemical on the target plant surface may then necessitate repeat applications of the chemical and/or application of the chemical at a higher concentration. As would be appreciated, this potentially compounds the problems associated with chemical wash-off and increases the potential for non-target environmental, plant and animal exposure to the chemical.

U.S. Pat. No. 5,724,765, to W. A. Wegner, describes a herbicide applicator comprising a pair of opposable jaws, each comprising an absorbent pad and a plurality of needles extending from each jaw into the pad. This device also comprises a herbicide reservoir which is connected by conduit to the absorbent pad on each jaw. When a plant is grasped by the jaws, the needles penetrate the surface of a plant. In this way, the herbicide, which is applied to the absorbent pads, coats the surface of the plant and then flows into holes produced in the plant tissue by the needles.

The device described in the Wegner patent does potentially lead to more effective utilization of herbicide, as wounding of the plant allows herbicide, from herbicide absorbent pads, to infiltrate the internal tissues of the plant. However, with the Wegner device, a significant amount of herbicide is still applied to the surface of the plant, via the absorbent pads. This surface-applied chemical is still prone to wash-off and thus can lead to exposure of non-target plants and animals to the chemical.

Methods for applying chemicals, such as pharmaceuticals, to animals are known. Such methods include injecting methods where a syringe is used to allow a pharmaceutical to directly flow from the syringe's reservoir down the barrel of a needle into the target tissue. It is also well known that injectable darts are used, which are based on the syringe technology.

Injecting methods may be suitable when a small number of specific target animals require an injectable pharmaceutical and when the target animal is accessible or lives in accessible media such as on land. However, injecting methods may be unsuitable where target animals are numerous, fast moving, interspersed with non-target plants and animals, remotely located, live in a medium such as below water or are out of reach and fast moving. In this case, injecting-based methods of pharmaceuticals may not be suitable for delivering the pharmaceutical to the target animals in an efficient and timely manner. Likewise, in situations where a large number of animals require specific doses of a pharmaceutical to be administered in a sterile manner, the syringe-style application devices have considerable limitations.

In light of the problems associated with injecting-based methods and devices, a number of direct-application or contact type chemical applicators have been developed in various attempts to address these problems.

For example, Patent Publication No. CN1415385 describes a miniature needle array sheet which supplies the pharmaceutical via said minipore array into the skin. Such devices can take the form of a transdermal patch to apply the pharmaceutical; however, such method and devices have limitations of applying pharmaceuticals to birds or water based animals, each patch has only a single use and used patches contribute to potentially toxic waste material within the environs when the patch is no longer attached to the target animal.

To overcome the deficiencies of, or to provide an alternative to, existing chemical methods and devices, the present invention provides a chemical applicator which substantially specifically delivers a chemical to the internal tissues of the living organism in this way, chemical coating of the surface of organisms would be minimized and thereby the potential for wash-off of the chemical, and subsequent exposure of non-target plants or animals would also be reduced. Substantially specific application of a chemical to the internal tissues of an organism would also potentially increase the effective utilization of a chemical, as the chemical could be delivered to the internal tissues of the organism where it is most effective, thus potentially decreasing the amount and/or concentration of a given chemical needed to achieve a desired outcome. Further, the application method can be rapidly reused to deliver the desired chemical to numerous organisms without the necessity to re-prime or re-sterilise the piercing elements.

Before turning to a summary of the invention, it must be appreciated that the above description of the prior art has been provided merely as background to explain the context of the invention. Accordingly, reference to any prior art in this specification is not, and should not be taken as, an acknowledgment or any form of suggestion that this prior art forms part of the common general knowledge in any country.

### SUMMARY OF THE INVENTION

One of the objects of the present invention is to provide a chemical applicator which uses a chemical application device and method for applying chemicals to the internal tissues living organisms, including the internal tissue of plants and animals which may reside in/on media such as air or land, or on/under water without contaminating the surrounding environment or non-target organisms.

The present invention provides a device for applying a chemical to a living organism, the device including:
A. one or more piercing elements for piercing the living organism and introducing the chemical into the living organism; and
B. a chemical reservoir for containing said chemical which comprises a body which is:
   i. normally sealed to encapsulate said chemical contained within said chemical reservoir; and
   ii. substantially impermeable to the chemical contained;

wherein the one or more of said piercing elements are engageable with one or more said reservoir body(s) such that said chemical is substantially specifically applied onto one or more of said piercing elements on exit of said chemical reservoir.

The present invention also provides a method of applying a chemical to a target using a device with one or more piercing elements and one or more chemical reservoirs, including the steps of:
a) containing said chemical in said chemical reservoirs;
b) disposing said piercing elements within said chemical reservoirs;
c) applying said chemical to said piercing elements within said reservoirs such that said piercing elements are chemically coated;
d) emerge said chemically coated piercing elements from said reservoir;
e) drawing said piercing elements through said target; and
f) releasing said chemical from said chemically coated piercing elements into said target; such that said delivery of said chemical is undertaken from said piercing elements are forced upon said target's surface to pierce said target when:
   I. said target is interspersed between said reservoir and one or more said piercing element(s); or
   II. one or more said piercing elements are extended outwards from said reservoir into said target.

The present invention may be used to deliver any chemical to a plant, animal or other living organism. Exemplary chemicals include, for example, herbicides, fungicides, growth regulators, fertilizers, genetic material, drugs, hormones, pheromones and other materials and the like which have an effect with inter- and intracellular administration to a living organism. However, in a preferred embodiment, the device of the present invention may be used to deliver a chemical to a plant.

It would be appreciated that the living organism's surface to be pierced is dependent on factors including:
1. chemical type,
2: application surface, and
3. access to the living organism's tissue to which the chemical is applied.

Chemical can be applied to animals, plants or other living organisms including lichen mosses or other animal species.

Referring to FIG. 1, the device of the present invention is predicated, in part, on one or more piercing elements 120 being engageable with a chemical reservoir 140 in order to effect the coating of one or more piercing elements 120 with the chemical. Once coated, these piercing elements may then be used to pierce living organism such as plant tissue and deliver the chemical to the internal tissues of the plant.

In a preferred embodiment, engagement of the one or more piercing 120 elements with the chemical reservoir 140 effects substantially specific application of the chemical onto the one or more piercing elements 120.

In this embodiment, because only the piercing elements are coated with the chemical, very little or substantially no chemical is applied to the surface of the living organism's tissue during delivery. In this way, the potential for wash-off of the chemical is reduced and substantially all of the chemical applied to the living organism is delivered to the internal tissues of the living organism, where it is likely to be of the greatest effectiveness.

In a further embodiment, the chemical reservoir 120 comprises a body 170 which is normally sealed to encapsulate the chemical contained within the chemical reservoir 140. The chemical contained within the chemical reservoir 140 may be any suitable type of formulation. For example, solid, liquid, suspension, paste or gel formulations are contemplated as being suitable for use in accordance with the present invention. Further, the chemical may be contained in wadding within the chemical reservoir, with the chemical reservoir's body's 170 being a membrane that protects the contained chemical from leaking and thus deteriorating, when in liquid would take the form of drying out. This chemical reservoir's body's 170 membrane is also piercable by one or more piercing elements 120 and functionally re-sealable on said piercing elements have disengaged from piercing said chemical reservoir's body's 170 membrane. Preferably still, the body is substantially impermeable to the chemical contained within the chemical reservoir.

More preferably, the piercing elements 120 are disposed within the chemical reservoir 140, such that one or more piercing elements 120 are normally coated with the chemical. Still preferably, the piercing elements 120 are chemically recoated on re-entry into the reservoir 140.

Preferably still, the chemical “reservoir” 140 may consist of a reservoir in the form of a removable “cartridge” which is preloaded with the chemical to release into the target living organism. The distinction between a “reservoir” and a “cartridge” is that a reservoir can be “fed” with the target chemical as well as “drained”. The “cartridge” in this arrangement can only be “drained” and therefore is replaceable.

The reservoir and the cartridge are one and the same since, in many embodiments, the reservoir may not require a separate cartridge. Therefore the reservoir/cartridge complex may be referred to as a reservoir alone hereafter. A preloaded reservoir, cartridge and/or reservoir/cartridge complex are, in some embodiments and arrangements, disposable and replaceable.

The reservoir is formed so that capillary attraction of the contained chemical draws the chemical towards any opening such as a piercing by the piercing elements 120. The opening may be formed by a variety of means including, for example, piercing the reservoir's surface by the piercing elements. Alternatively, the reservoir may contain material, which by capillary attraction, draws up the contained chemical to an opening of the reservoir. Such material may take the form of a bundle of fibers or a loosely twisted, braided, or woven cord, tape, or tube usually of soft spun cotton threads in the form of wadding. This embodiment may or may not require an external membrane surrounding the reservoir's body 170. This preloaded reservoir overcomes problem relating to the filling and the re-filling of chemical containers with potentially hazardous and/or poisonous chemicals by unaware consumers.

Preferably further, the reservoir's 140 wadding to be used to hold the chemical and assist its transfer onto one or more piercing elements 120. Such wadding in various arrangements is also selectable to provide a reaction force for ‘spring back’ of the piercing elements when entering the reservoir after it retracts when pressed against an organism such as a weed.

Further preferably, retraction of one or more of said piercing elements 120 from said chemical reservoir 140 effects a substantial re-sealing of the reservoir's surface so not enabling leakage of further chemical. The stopping of release of chemical leakage is partially due to one or more of the following:
1. the surface tension of the chemical, so that it remains held at the pierced opening;
2. the narrow pierced surface made by one or more piercing elements as they enter and/or re-enter the reservoir is of a size to limit leakage; and
3. the chemical in the reservoir 140 is contained in the wadding, so that when one or more piercing elements 120 wipe past the wadding, the chemical is attached to the piercing elements; however, in the absence of the piercing elements 120 the chemical within the reservoir 140 is not in a form to leak from an opening in the reservoir's surface.

In the absence of chemical containing wadding, the surface of the reservoir 140 is treated with a sealant membrane to avoid transfer of chemical, such as a chemical onto the leaf except by way of the piercing element 120. This membrane is to minimise leakage in its many forms including evaporation.

The piercing element 120 is moulded into a cap 190 of the chemical applicator or, alternatively, is a separate element that is bonded/fused/attached to the cap (therefore, taking the form of an alternate material including a metal pin, blade, barb or other suitable material).

The cap provides extra ‘lead-in’ by forming, for instance, a mouth to guide the leaf such that the lead-in cap can is selectable for various vegetation types. For example, in some arrangements, the cap 190 is interchangeable depending on piercing elements and cap dimensions required. That is, the cap is selectable for target organisms. For example, woody stemmed weeds such as lantana may require a wider mouthed cap with a greater gap to selectively position the stem between the piercing elements 120 and the chemical reservoir 140, whereas a thin grassy weed may require a narrower mouth and smaller number of piercing elements.

The number and form of the piercing elements may be moulded or inserted into the cap as selectable depending on the type of chemical application required. Further, the piercing element(s) may be designed to produce a recognisable mark on the vegetation to indicate that treatment has occurred. Additionally, the reservoir may include a dye with the chemical (such as a herbicide) to mark the application of the chemical as obvious and to show that the chemical applicator is operational.

The cap has a non-mandatory optional component in that it takes a form of a closeable cap as shown in FIG. 6. The chemical applicator in its various embodiments includes the cap plus, optionally, a closable portion of the cap to prevent drying out of the chemical reservoir when it takes the form of chemical containing wadding. The optional closable cap provides an additional barrier between the user's fingers and the chemical.

Further preferably, the piercing elements are cleansed on entering the chemical reservoir 140. More preferably, the piercing elements 120 emerge from the chemical reservoir 140 with a chemical coating. Still preferably, the piercing elements are re-cleansed on re-entering into the chemical reservoir 140.

Preferably, the piercing elements are held in the cap or directly contained and raised from a first member 110, which is pivotal on an axis to a second member 130, which respectively holds, directly or indirectly, the chemical reservoir 140. This facilitates, among other things, ready handling via handles 160 and use of the device. The first and second members may be attached through one or more pivot, fulcrum or axis points (collectively referred to as pivot points 150), and these pivot points may be arranged such that the device adopts, for example, a ‘tong’ type configuration (also referred to as a “jaws” type configuration) wherein a pivot point 150 is placed proximal to, or at one end of the first and second members.

Preferably, the first and the second members include handles 160, wherein the handles are actuated to bring one or more piercing elements 120 and the chemical reservoir 140 into engagement. In use, these handle(s) may be manipulated by a user to bring the piercing elements and chemical reservoir into, and/or out of, engagement. The handle(s) 160 may be placed anywhere along the first and second members that is compatible with placing pressure on the actuator 180. For example, when the placement of the pivot point is such that the device adopts a ‘tong’ or ‘jaw’ type configuration, the handle(s) are preferably located on the first and/or second members intermediate to the pivot point, so that when held the actuator 180 can have force applied to lower the piercing elements through the tissue of a held organism into the chemical reservoir 140 and then, via the resilient bias of the actuator 180, return the piercing element to its starting position with release of force on the actuator, so that on return to the piecing element's starting position the tissue is applied with chemical.

The engagement of the chemical reservoir 140 against the living organism is further facilitated by engagement with the piercing elements 120, which is enabled to initiate a secondary sealing function, reducing potential loss of chemical to the environment. While the invention will function advantageously against the prior art without such a seal under certain environmental conditions, a seal may provide an advantage over the prior art.

In a second form of the invention, the one or more piercing elements are disposed within the chemical reservoir, such that the one or more piercing elements are normally coated with the chemical; and wherein the one or more piercing elements may be extended outwardly from the chemical reservoir to apply the chemical to a living organism and withdrawn back into the chemical reservoir to effect re-sealing of the chemical reservoir.

The device's actuator 180, with actuation of the actuator, effects extension of the piercing elements into the chemical reservoir with finer control than via the handles alone. That is, the actuator, when a user inserts pressure, for example, via their fingertip onto the actuator, allows appreciation via movement of the piercing elements to and from the chemical reservoir due to greater sensitivity than that enabled by moving the handles alone. In a more preferred embodiment, the actuator comprises the application of force along the longitudinal axis of the handle to allow the insertion of the piercing elements through the organism into the chemical reservoir which the handles hold the organism in position.

Further preferably, the piercing elements 120 are cleansed on entering the chemical reservoir 140. More preferably, the piercing elements emerge from the chemical reservoir with a chemical coating. Still preferably, the piercing elements are re-cleansed on re-entering into the chemical reservoir.

Still further preferably in alternate embodiments, there is an electronic detection method such as detector, in the form of an infrared or alternate detector, detects entry of a weed in between the jaws of the device and the actuator is automatically activated making it possible to treat the weed by just guiding it into the cavity between the two jaws of the device.

Once the presence of the weed is detected by the virtue of the break in the exemplary infra-red beam, the piercing element is automatically depressed and the function of the device in treating the weed is automatically performed.

The present invention also provides a method of applying a chemical to a living organism, using a device with one or more piercing elements and one or more chemical reservoirs, including the steps of:
a) containing the chemical in the chemical reservoir;
b) disposing the piercing elements within the chemical reservoir;
c) applying the chemical to the piercing elements within the reservoir such piercing elements are chemically coated;
d) emerge the chemically coated piercing elements from the reservoir;
e) drawing the piercing elements into the living organism; and
f) releasing the chemical from the chemically coated piercing elements into the living organism.

Preferably, in one form of the invention, the delivery of the chemical from the piercing elements into the organism occurs when the organism is interspersed between the reservoir and the piercing element which pierces the organism's surface.

Preferably, in another form of the invention, the delivery of the chemical is undertaken when the piercing element 120, biased within the reservoir, is forcibly extended outwards from the reservoir into the organism.

The present invention also provides a device for applying a chemical to a plant, the device including:
a) one or more piercing elements for piercing the plant and introducing the chemical into the plant; and
b) a chemical reservoir for containing the chemical; wherein the one or more of the piercing elements are engageable with one or more reservoirs such that the chemical is substantially specifically applied onto one or more of the piercing elements.

The present invention also additionally provides a method of applying a chemical to a plant, using a device with one or more piercing elements and one or more chemical reservoirs, including the steps of:
a) containing the chemical in the chemical reservoir;
b) disposing the piercing elements within the chemical reservoir;
c) applying the chemical to the piercing elements within the reservoir such that the piercing elements are chemically coated;
d) emerge the chemically coated piercing elements from the reservoir;
e) drawing the piercing elements into the plant; and
f) releasing the chemical from the chemically coated piercing elements into the plant.

The present invention further provides also a device for applying a chemical to an animal, the device including:
a) one or more piercing elements for piercing the animal and introducing the chemical into the animal; and
b) a chemical reservoir for containing the chemical; wherein the one or more of the piercing elements are engageable with one or more reservoirs such that the chemical is substantially specifically applied onto one or more of the piercing elements.

The present invention also provides also a method of applying a chemical to an animal, using a device with one or more piercing elements and one or more chemical reservoirs, including the steps of:
a) containing the chemical in the chemical reservoir;
b) disposing the piercing elements within the chemical reservoir;
c) applying the chemical to the piercing elements within the reservoir such that the piercing elements are chemically coated;
d) emerge the chemically coated piercing elements from the reservoir;
e) drawing the piercing elements into the animal; and
f) releasing the chemical from the chemically coated piercing elements into the animal.

Throughout this specification, unless the context requires otherwise, the word “include”, or variations such as “includes” or “including”, will be understood to imply the inclusion of a stated element or integer or group of elements or integers but not to the exclusion of any other element or integer or group of elements or integers.

### BRIEF DESCRIPTION OF THE FIGURES

Having briefly described the general concepts involved with the present invention, exemplary preferred embodiments of the chemical applicator of the present invention will now be described with reference to the following figures:

FIG. 1 shows a cross-sectional side view of a chemical applicator according to one preferred embodiment of the invention.

FIG. 2 shows a top view of the chemical applicator.

FIG. 3 shows a side view of a chemical applicator.

FIG. 4 shows an end view containing the cap on the chemical applicator according to one preferred embodiment of the invention.

FIG. 5 shows a perspective cross-sectional side view of a chemical applicator according to one preferred embodiment of the invention in an open position.

FIG. 6 shows a perspective cross-sectional side view of a chemical applicator according to one preferred embodiment of the invention in a closed position.

### DESCRIPTION OF THE PREFERRED EMBODIMENTS

It is to be understood that the following description is for the purpose of describing particular embodiments only, and is not intended to be limiting with respect to the above description.

The term “organism” and “living organism” is used to include “any living structure; such as a plant, animal, fungus or bacterium, capable of growth and reproduction” as defined by Chambers online Reference.

It would be appreciated that the living organism's surface to be pierced is dependent on factors including;
1. chemical type, and
2. the internal tissue type to which it is applied.

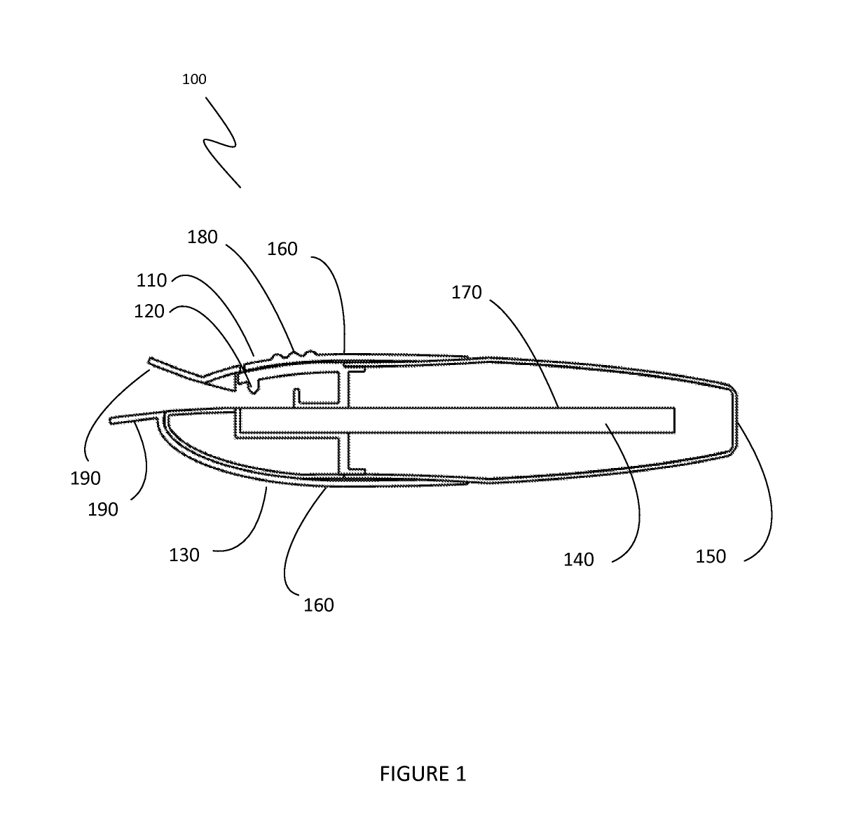

Additionally, the surface tissue my require piercing due to the non-absorbent nature of the external tissue. For example, a leaf's cuticle may be very waxy, a weed's stem may be woody or an organism's exoskeleton may be tough. Therefore, as shown in FIG. 1, the cap (190) is chosen to lead the target tissue into the cap's mouth with a desired alignment of the piercing element (120) to the target tissue to be pierced. Here, the required tension to pierce the tissue in a desired manner is felt by the users as feedback when placing a force such as closing a grip on the handles (160).

Applied surfaces such as plant surfaces include any other surface contemplated including applications to lichen, mosses, and animal species.

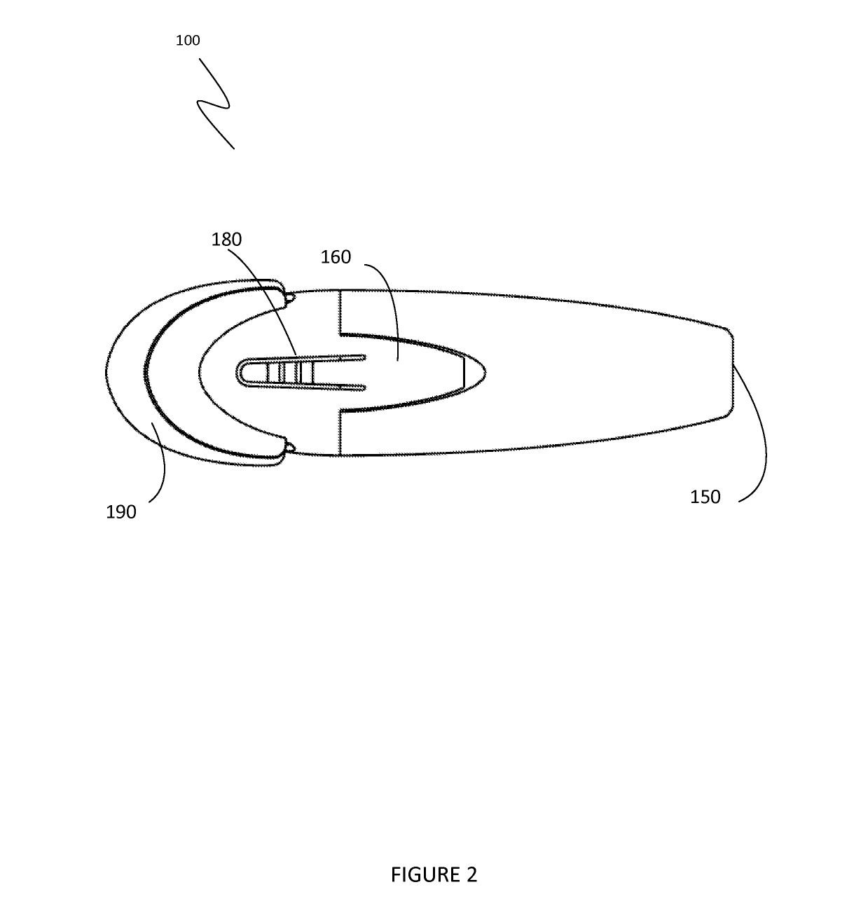

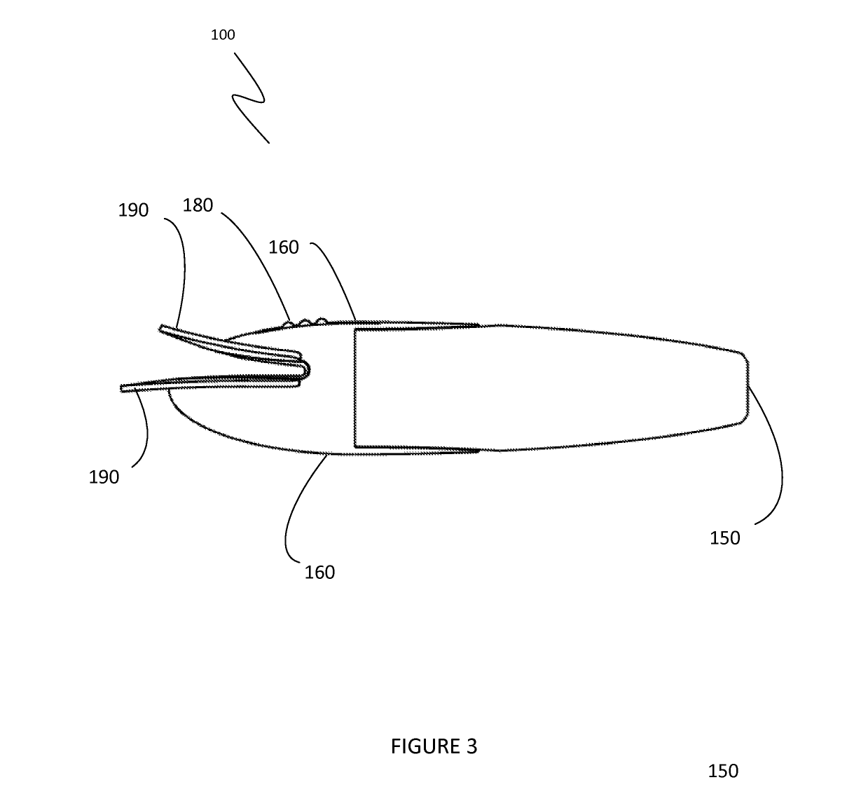

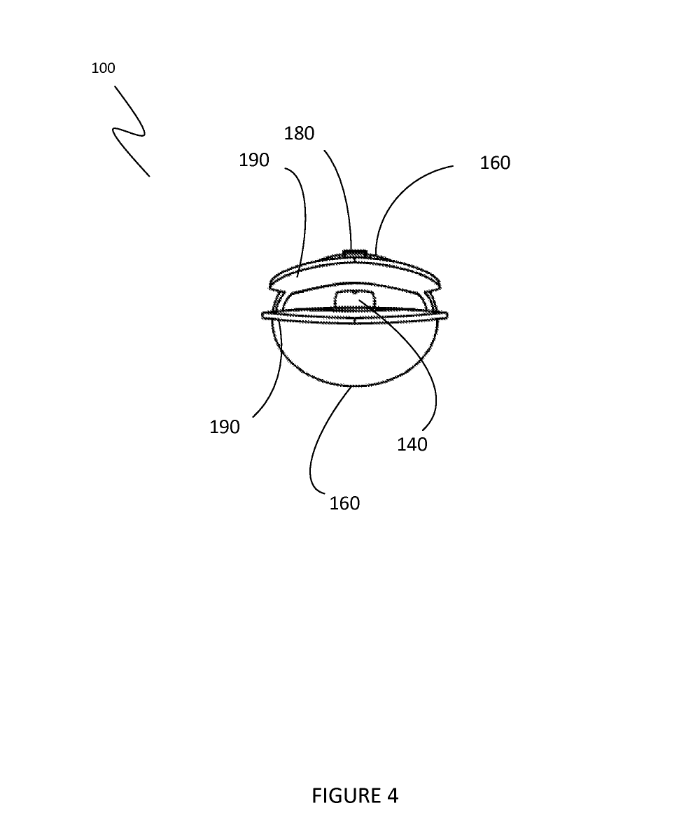

As shown in FIGS. 1 to 6, the device of the present invention is predicated, in part, on the piercing elements 120 (not shown in FIGS. 2, 3 and 4, since in FIGS. 3 and 4 the piercing element is not actuated or forced into an engageable position, whereas in FIG. 2 this is a view from above and not a cross sectional view) being engageable with the chemical reservoir 140 (not shown in FIGS. 2 and 3, since these are not cross sectional views) in order to effect the coating of the piercing elements with the chemical. Once coated, these piercing elements may then be used to pierce living organism's tissue such as plant tissue to deliver the chemical. This coating of the piercing elements (120) by engagement with the chemical reservoir (140) is enabled by a variety of methods, including:
1. charging the piercing elements (120) by engagement with chemical reservoir (140) with the cap's mouth empty such that there is no organism present;
2. passing the piercing elements (120) through an organism in the cap's mouth into the chemical reservoir (140) and returning the charged piercing elements (120) back through the organism to their disengaged position; and/or
3. passing the piercing elements (120) through the chemical reservoir (140) into the organism present in the cap's mouth.

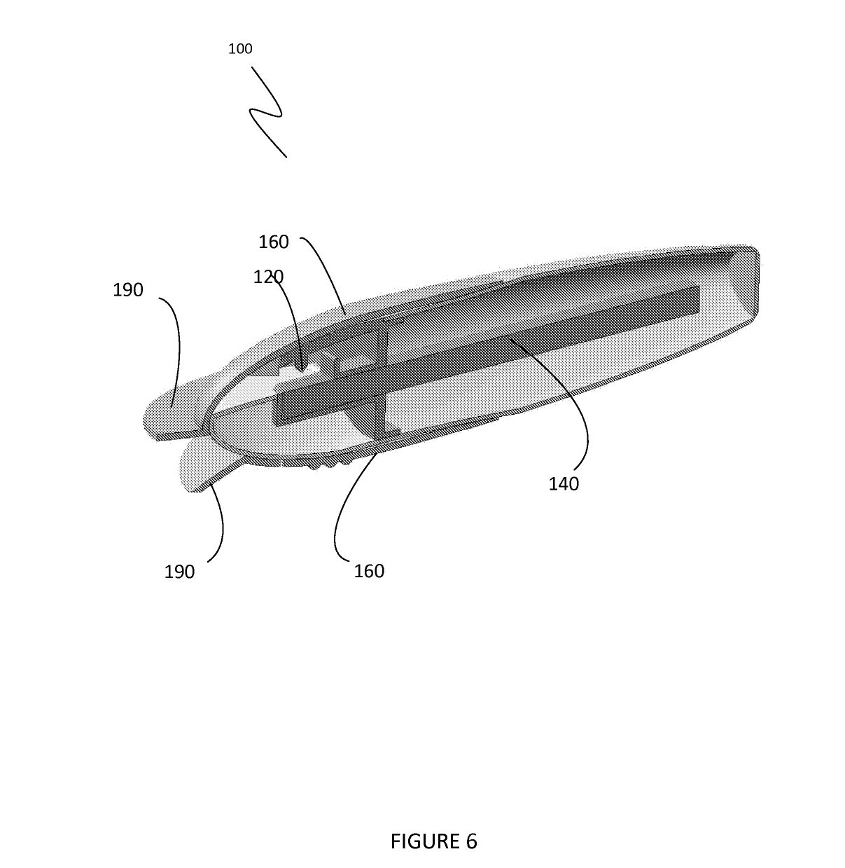

Referring to FIG. 4, the tip of this chemical applicator device in its preferred embodiment consists of the reservoir 140 protruding into the cap's 190 mouth, so that it is positioned under the piercing elements 120 (not shown since piercing elements are not forced down and the mouth is of the cap is open). These piercing elements are enabled to extend from within the end cap (that seals the body of the chemical applicator) once they are pressured down by squeezing the actuator 180 whilst holding the handles 160 and/or pressing the actuator 180 directly. The cap's mouth is covered by a reversibly slidable flexible boot when closed as shown in FIG. 6.

In an alternate embodiment when the chemical applicator's boot is in a retracted (open) position, the weed can be received by the cap's mouth. Additionally, when the boot is opened, the chemical reservoir 140 retracts back into the applicator's body so as to expose the piercing elements 180. These piercing elements 180 normally reside within the chemical reservoir 140, which comes forward when the boot is closed.

The term chemical is used to include herbicides, fungicides, growth regulators, fertilizers, genetic material, drugs/pharmaceuticals, small amino acid chains such as DNA and RNA, hormones, pheromones, viruses and any other substance contemplated which would have a beneficial effect with the inter- and intracellular administration to a living organism.

The preferred embodiments described are for use in delivering chemicals, for example, to noxious weeds strewn among desired plants, including those with woody stems such as Lantana, vines such as Kudzu as well as common ground weeds, such as thistle.

Another application of the preferred embodiment enables other forms of chemicals, aimed at enhancing the performance of the desired targeted plant for purposes such as fertilizing, applying pesticides to repel or kill harmful pests, fungicides to eliminate potentially harmful effects of fungal growth on or near desired plants and the like, to be applied.

Therefore, the embodiments described show one or more chemical reservoirs and one or more piercing elements. However, other arrangements can be envisaged by the person skilled in the art. For example, reducing the piecing elements and chemical reservoirs down to only one chemical reservoir and one accompanying piercing element would enable the preferred embodiment to be used for delivering specific chemical doses to target sea or animal life such as fish, mammals or poultry.

A device for applying a chemical to a living organism the said device including:

a) one or more piercing elements for piercing the surface of the living organism and introducing the chemical to the internal tissues of the living organism; and

b) a chemical reservoir for containing the chemical;

c) wherein the one or more of the said piercing elements are engageable with the reservoir in a manner such that chemical is substantially specifically applied onto the one or more of the said piercing elements.

Also, as set out above, in one preferred form of the invention, engagement of the one or more piercing elements with the chemical reservoir effects release of the chemical from the chemical reservoir substantially only onto the one or more piercing elements, and disengagement of the one or more piercing elements from the chemical reservoir effects re-sealing of the chemical reservoir. A particularly preferred embodiment of this form of the invention is shown in FIG. 1

In this embodiment, the chemical applicator 100 comprises a first member 110 carrying a plurality of piercing elements 120 extending outwardly therefrom. These piercing elements 120 are for piercing the surface of a living organism and introducing a chemical to the internal tissues of the living organism. The chemical applicator 100 further comprises a second member 130 which comprises a chemical reservoir 140 containing a chemical, for example, such as a herbicide.

The chemical reservoir 140 is modular and, in some embodiments and arrangements, is enabled to be detached from the second member 130 to facilitate replacement of the chemical reservoir once the chemical therein has been exhausted. In this way, it is unnecessary to decant chemical into the chemical reservoir 130 from another vessel in order to use the chemical applicator 100. Thus, the chance of exposure of the user or other non-target living organisms or animals to the chemical is further reduced.

Preferably, the first Member 110 is pivotally, springingly or flexibly attached to the second member 130 such that the piercing elements 120 and chemical reservoir 140 may be brought into, and/or out of, engagement via placing force on the handles 160 (via a manual or automatically/mechanically mediated method) and then releasing, such that the springingly engagement of the piercing elements 120 into the organism and/or the chemical reservoir 140 is followed by a returning action back to a position where the piercing elements are disengaged from the penetrating position. In some arrangements, this returning action may take the form of a recoiling action to provide feedback to the user that the piercing elements are disengaged.

In a particularly preferred embodiment, as shown in FIG. 1, the first member 110 and second member 130 are coupled to each other about an axis point, referred hereafter as a pivot 150. The pivot 150 allows the first member 110 and second member 130 to move with respect to each other and bring the piercing elements 120 and chemical reservoir 140 into, and/or out of, engagement.

As set out above, the first and/or second members (110, 130) preferably further comprise a handle 160, wherein the handle(s) 160 may be manipulated by a user to bring the piercing elements 120 and chemical reservoir 140 into, and/or out of, engagement.

For example, with reference to the particularly preferred embodiment in FIG. 1, each of the first member 110 and second member 120 further comprise handles 160. These handles are positioned such that movement of the handles together causes the piercing elements 120 and chemical reservoir 140 to move toward each other and ultimately move into engagement with each other, and separation of the handles 160 causes the piercing elements 120 and chemical reservoir 140 to disengage and separate.

In order to apply a chemical to a living organism, the handles 160 are initially moved apart, thus separating the piercing elements 120 from the chemical reservoir 140. A target living organism is then placed between the separated piercing elements 120 and chemical reservoir 140. Once a living organism is in position, the handles 160 are moved together, thus causing the piercing elements 120 to penetrate through the living organism and ultimately engage with the chemical reservoir 140. The engagement process may also serve a secondary sealing function of reducing the exposure of the piercing elements to the environment.

As set out above, the piercing elements 120 on the first member 110 are engageable with the chemical reservoir 140 on the second member 130 to release chemical from the reservoir 140 onto the piercing elements 120.

Once the piercing elements 120 are coated, the handles 160 may be separated, thus causing disengagement of the piercing elements 120 from the chemical reservoir 140. Once the piercing elements 120 are disengaged, the further escape of chemical from the chemical reservoir 140 is prevented. This is due to a number of reasons including:
1. the chemical reservoir's 140 pierced surface resealing;
2. the surface tension of the chemical being too great to enable leaking; or
3. osmotic pressure holding the chemical with the chemical reservoir 140.

When the handles 160 are further separated due to the springback or recoil of the handles, the piercing elements 120 are drawn back through the tissue of the living organism and, accordingly, deliver the chemical carried on the coated piercing elements 120 to the internal tissues of the living organism.

Any chemical remaining on the piercing elements 120 after being drawn through the living organism tissue may be re-applied to the same living organism or a different living organism by placing further living organism tissue in the space between the separated first and second members 110 and 130 and bringing the handles 160 together again. This action would also eventually cause further application of chemical to the piercing elements 120 when they engage with the chemical reservoir 140, as described above.

The piercing elements 120 are cleansed on re-entering the chemical reservoir 140. Such cleansing is achieved by the wiping of the piecing elements 120 along said chemical reservoir's 140 containing wall in the form of an outer membrane and/or the internal wadding (if present).

Such cleansing and re-cleansing is achieved by the wiping of the piecing elements as the handle(s) are manipulated by a user to bring the piercing elements 120 and chemical reservoir 140 into, and/or out of, engagement.

In a second form of the invention, the one or more piercing elements are disposed within a chemical reservoir, such that the one or more piercing elements are normally coated with the chemical; and wherein the one or more piercing elements may be extended outwardly from the chemical reservoir to apply the chemical to a living organism and withdrawn back into the chemical reservoir to effect sealing of the chemical reservoir.

In use, a user may then grip the handles 160 and carry the device to a living organism, for example, a broadleaf weed such as carpetweeds. The piercing elements 120 may then be manually, automatically or mechanically positioned over the living organism by sliding the living organism through the mouth of the cap 190. Once the device has been positioned over a living organism, the user exerts a force on the handles 160, which, as described above, effects compression of the chemical reservoir 140 and extension of the piercing elements 120 together with the living organism, as described above. The extended piercing elements 120 penetrate the tissue of the organism and deliver the chemical thereto. When the force on the handles 160 is released, the handles 160 bias the chemical reservoir 140 and piercing elements 120 into the expanded configuration, which, in turn, causes the piercing elements 120 to retract back from the chemical reservoir 140.

The piercing elements 120 are cleansed on entering said chemical reservoir 140. Further, the piercing elements 120 are re-cleansed on re-entering the chemical reservoir 140. Such cleansing and re-cleansing is achieved by the wiping of the piecing elements into said chemical reservoir 140. The cycle of cleansing and re-cleansing follows the cycle of the user exerting a force manually, automatically or mechanically along the length of the handles 160, followed by the release of the force causing the piercing elements 120 to retract back into the chemical reservoir 140.

With chemical use, a preferred embodiment of the chemical applicator's clasping face holding the piercing elements 120 can be as small as 1 square micron to many square meters, incorporating one or many piercing elements as required, depending on the target organism's chemical application requirements. The cap may be substituted for different sized and number of piercing elements 120 on the clasping face. The upper and lower limits are not however so limited and could be vastly different, with the optimum size being determined by factors including but not limited to, the chemical type, form and target organism type.

FIG. 4 shows by way of example the method of applying a chemical to a living organism, including a plant or an animal, with one or more piercing elements and one or more chemical reservoirs, including the steps of:
a) containing the chemical in the chemical reservoirs;
b) disposing the piercing elements into the chemical reservoirs;
c) applying the chemical to the piercing elements within the reservoirs such that the piercing elements are chemically coated;
d) emerge the chemically coated piercing elements from the reservoir;
e) drawing the piercing elements through the organism; and
f) releasing the chemical from the chemically coated piercing elements into the organism.

The method of delivery, according to the first embodiment of the invention, of the chemical from the piercing elements to a plant, animal or living organism occurs when the organism is interspersed between the piercing elements 120 and reservoir 140. The piercing element which pierces the organism's surface as the handles 160 are forced together. Here the piercing element(s) 120, in the case of a plant, extend through the plant into the chemical reservoir, where piercing element(s) are coated with the chemical contained in the reservoir 140. The members are subsequently separated and the piercing element(s) pass back through organism and the chemical is released from the piercing elements into the organism's body tissue. The cycle can then repeat to re-apply the chemical onto the piercing elements where necessary.

A similar cycle can also be repeated when the organism's tissue is too thick to allow the piercing element(s) to pass completely through the tissue. In with the absence of the organism interspersed between the members, when the members are closed the piercing element(s) are coated with the chemical contained in the chemical reservoir. Once the piercing elements are coated and the members are opened and reclosed with an organism, such as an animal or a plant, interspersed, the chemical can be applied via the recoated piercing element(s) insertion into the organism's tissue when the members are forced closed.

The cycle of re-coating the piercing elements can take place once again when the body tissue is too thick to allow the piercing elements and chemical reservoirs to engaged, then the members can be released, allowing the organism to vacate the space between the members. Hence, the piercing elements and reservoir can now engage to allow re-coating of the piercing elements with the chemical.

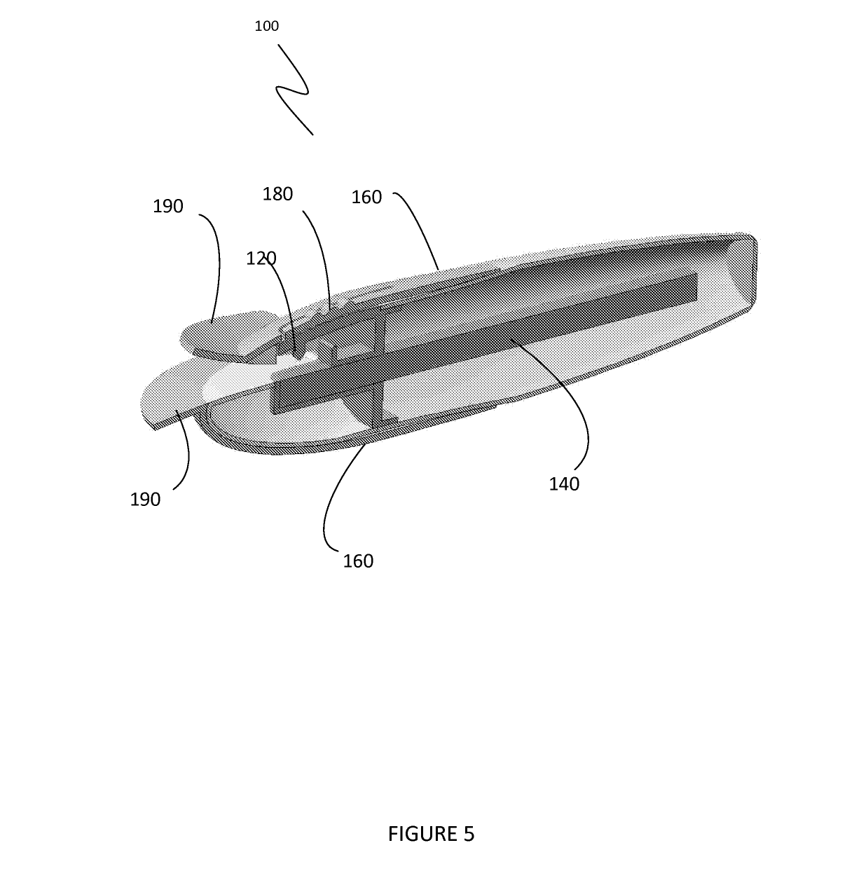

FIG. 5 shows by way of example the method of applying a chemical, according to the one embodiment of the invention which enables the delivery of the chemical to take place when the piercing element, normally biased within the reservoir, being forcibly extended outwards from the reservoir into the animal's contacting surfaces underlying tissue.

As would be appreciated, the present invention encompasses manually-operated embodiments which may be used, for example, in small scale domestic, farming or nursery settings. However, it should also be understood that the present invention also encompasses larger scale, and optionally automated, embodiments which may be used, for example, in market garden, broadacre agriculture farming and aquaculture applications. Furthermore, the invention may also be used in conjunction with automated tissue sensing systems (for examples, see the publication of Steward and Tian, Transactions of the ASAE 42(6): 1897-1910, 1999).

While plant applications have been exemplified in this description, those skilled in the art will recognise that the invention could also administer chemicals not only to plant tissue but also to animal tissue or any living tissue. Those skilled in the art will further appreciate that the invention described herein is susceptible other variations and modifications other than those specifically described. It is to be understood that the invention includes all such variations and modifications. The invention also includes all of the steps or features referred to, or indicated in this specification, individually or collectively, and any and all combinations of any two or more of the steps or features.

Also, it must be noted that, as used herein, the singular forms “a”, “an” and “the” include plural aspects unless the context already dictates otherwise. For example “a piercing element” may be a single element or may include a plurality of elements.

Future patent applications may be filed in Australia or overseas on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the invention or inventions inherent in the present disclosure.

What is claimed is:

1. A device for applying a chemical, said device including:
A. one or more piercing elements for piercing said living organism and introducing said chemical into said living organism; and
B. a chemical reservoir for containing said chemical which comprises a body; wherein the one or more of said piercing elements are engageable with one or more said reservoir body(s) such that said chemical is substantially specifically applied onto one or more of said piercing elements.

2. The device of claim 1 wherein said piercing elements are cleansed on entering into said chemical reservoir.

3. The device of claim 1 wherein said piercing elements are re-cleansed on re-entering into said chemical reservoir.

4. The device of claim 1 wherein said piercing elements are raised from a first member which is flexibly attached to a second member which houses said chemical reservoir.

5. The device of claim 4 wherein said first and said second members includes one or more handles, wherein said handles are actuated to bring one or more said piercing elements and said chemical reservoir into reversibly biased engagement.

6. The device of claim 1, including an actuator, wherein actuation of said actuator effects reversibly biased engagement of said piercing elements into said chemical reservoir.

7. The device of claim 6 wherein said actuator comprises a handle and actuation of said actuator comprises the application of force along the longitudinal axis of said handle.

8. The device of claim 6 wherein said chemical reservoir includes a chemical which is releasable onto said piercing elements in response to the engagement of one or more said piercing elements.

9. The device according to claim 8 wherein a seal is created in said chemical reservoir when one or more said piercing elements sealingly engage and withdraw from said chemical reservoir.

10. The device of claim 8 wherein said piercing elements are cleansed on entering said chemical reservoir.

11. The device of claim 8 wherein said piercing elements emerge from said chemical reservoir with a chemical coating.

12. The device of claim 8 wherein said piercing elements are re-cleansed on re-entering said chemical reservoir.

13. The device of claim 8 wherein said piercing elements are chemically recoated on re-entry into said reservoir.

14. The device of claim 8 said seal is created in said chemical reservoir when one or more said piercing elements sealingly engage with said chemical reservoir.

15. A method of applying a chemical to a target using a device with one or more piercing elements and one or more chemical reservoirs, including the steps of:
a) containing said chemical in said chemical reservoirs;
b) disposing said piercing elements within said chemical reservoirs;
c) applying said chemical to said piercing elements within said reservoirs such that said piercing elements are chemically coated;
d) emerge said chemically coated piercing elements from said reservoir;
e) drawing said piercing elements through said target; and
f) releasing said chemical from said chemically coated piercing elements into said target; such that said delivery of said chemical is undertaken from said piercing elements are forced upon said target's surface to pierce said target when:
I. said target is interspersed between said reservoir and one or more said piercing element(s); or
II. one or more said piercing elements are extended outwards from said reservoir into said target.

16. The method in claim 15 wherein delivery of said chemical is undertaken when said target is interspersed between said reservoir and said piercing element such that a sealingly engagement is created between the contacting surfaces.

17. The method in claim 16 wherein delivery of said chemical is undertaken when said piercing element, biased within said reservoir, is forcibly extended outwards from said reservoir into said target.
